(19) United States
(12) Patent Application Publication
(10) Pub. No.: US 2017/0095428 A1
(43) Pub. Date: Apr. 6, 2017

(54) AGENT FOR IMPROVING GLUCOSE TOLERANCE DISORDER

(71) Applicants: National Institute of Advanced Industrial Science and Technology, Tokyo (JP); Nisshin Seifun Group Inc., Tokyo (JP); Oriental Yeast Co., Ltd., Tokyo (JP)

(72) Inventors: Katsutaka OISHI, Ibaraki (JP); Nanako ITOH, Ibaraki (JP); Saori YAMAMOTO, Ibaraki (JP); Shin-ichi FUKUDOME, Saitama (JP); Yosuke KIKUCHI, Saitama (JP); Kimiko OKITA, Tokyo (JP)

(21) Appl. No.: 15/311,693

(22) PCT Filed: Jun. 17, 2014
(86) PCT No.: PCT/JP2014/066007

§ 371 Date: Nov. 16, 2016

(51) Int. Cl. A61K 31/047; A23L 2/52; A23K 50/40; A23L 33/105; A61K 36/899; A23K 20/10

(52) U.S. Cl. CPC A61K 31/047; A61K 36/899; A23K 20/10; A23K 50/40; A23L 33/105; A23L 2/52; A61K 2236/33; A23V 2002/00

(57) ABSTRACT

An impaired glucose tolerance ameliorative agent containing a peak component of partition chromatography of an gramineous plant seeds alcohol extract. The peak component preferably contains an alkylresorcinol (AR) mixture containing a plurality of alkylresorcinols (ARs). The AR mixture preferably contains (1) an AR of general formula (I) wherein R₁ is a C15 saturated or unsaturated alkyl group, (2) an AR of formula (I) wherein R₁ is a C17 saturated or unsaturated alkyl group, (3) an AR formula (I) wherein R₁ is a C19 saturated or unsaturated alkyl group, (4) an AR of formula (I) wherein R₁ is a C21 saturated or unsaturated alkyl group, (5) an AR of formula (I) wherein R₁ is a C23 saturated or unsaturated alkyl group , and (6) an AR of formula (I) wherein R₁ is a C25 saturated or unsaturated alkyl group.

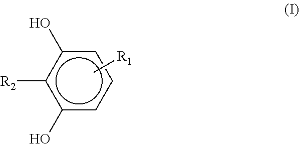

R₁ is C15-C25 saturated or unsaturated alkyl. R₂ is hydrogen or methyl.

### TECHNICAL FIELD

This invention relates to an impaired glucose tolerance (IGT) ameliorative agent which is effective in reducing a blood glucose level increase in humans and animals and exhibits prophylactic and therapeutic effects on IGT including hyperglycemia. Impaired glucose tolerance (IGT) is a condition in which fasting blood glucose levels are higher than normal. IGT occurs in not only humans or animals diagnosed as diabetes mellitus (hereinafter simply “diabetes”) but also those who are not yet diagnosed as diabetes (also called pre-diabetics).

### BACKGROUND ART

In recent years, persons with diabetes or IGT (pre-diabetes) have been increasing because of westernization of diets, chronic lack of physical exercise, and the like. Prevention and treatment of diabetes are extremely important because diabetes can develop complications, such as diabetic neuropathy (e.g., numbness and pain), cataract, diabetic retinopathy, arteriosclerosis, diabetic nephropathy, and diabetic gangrene and may lead ultimately to death. In order to prevent and/or treat diabetes (or IGT), lifestyle modifications are desirable but not easy to achieve. Diabetes treatment includes drug therapy for controlling blood insulin concentrations, such as insulin injection and oral administration of a hypoglycemic agent. However, drug therapy is associated with side effects. Dietary therapy and exercise therapy involve the difficulty for patients to comply with given instructions. Additionally, the occurrence of diabetes and IGT is not confined to humans and has recently posed similar problems in pet animals, such as cats and dogs.

In the light of these circumstances, attempts have been made to prevent and treat diabetes (IGT) using a naturally occurring component that exhibits various effects with no risk of potential side effects. For example, Patent Literature 1 below describes an amylase inhibitor derived from wheat as being effective in reducing a blood glucose level increase or controlling insulin secretion. Patent Literature 2 below discloses a preventive and ameliorative agent for hyperlipidemia, diabetes, obesity, hypertension, and like diseases that contains a peroxisome proliferator-activated receptor (PPAR) ligand agent containing as an active ingredient an extract of a gramineous plant.

It is known that a synthetic alkylresorcinol or a derivative thereof and an alkylresorcinol-containing extract from the seed coat of cereal crops, such as wheat and rye, and the seed coat of nuts, such as cashew nut, have anti-obesity effect, antidiabetic effect, and the like. For example, Patent Literature 3 below discloses, as an obesity preventive agent, a glycerophosphoric acid dehydrogenase inhibitor containing a 5-alkylresorcinol having 14 to 16 carbon atoms in the alkyl moiety as an active ingredient. Patent Literature 4 below teaches that an olefin-substituted compound having a specific core moiety is effective in treating diabetes (see claim 12 of Patent

Literature 4), presenting resorcinol as an example of the core moiety (see page 8 of Patent Literature 4). Patent Literature 5 describes that induction of secretion of adiponectin, one of adipocytokines (physiologically active substances secreted from fat tissues), is effective in prevention and treatment of arteriosclerosis, obesity, diabetes, and so on and that a specific component extracted from a specific plant induces secretion of adiponectin, presenting a resorcinol derivative as an example of the specific component.

### CITATION LIST

### Patent Literature

Patent Literature 1: JP 7-41499A

Patent Literature 2: JP 2004-161656A

Patent Literature 3: JP 6-100440A

Patent Literature 4: JP 8-508242A

Patent Literature 5: JP 2005-68132A

### SUMMARY OF INVENTION

As a result of intensive investigations, the inventors of the present invention have found that a peak component of partition chromatography of an alcohol extract from the seed of a gramineous plant contains a mixture containing a plurality of specific alkylresorcinols and that the mixture (hereinafter referred to as a specific alkylresorcinol mixture) is effective in prevention and treatment of IGT.

Based on the above finding, the invention provides an impaired glucose tolerance ameliorative agent containing as an active ingredient a peak component of partition chromatography of an alcohol extract of a gramineous plant seed.

### BRIEF DESCRIPTION OF DRAWINGS

FIG. 1 is a graph showing the blood glucose level lowering effect of the IGT ameliorative agent of Example according to the invention in a confirmatory test for blood glucose level lowering effect (ad lib. ingestion).

FIG. 2 is a graph showing the IGT ameliorative effect of the IGT ameliorative agent of Example according to the invention in a glucose tolerance test.

FIG. 3 is a graph of AUC_{0-120 min} values obtained in the glucose tolerance test.

FIG. 4 is a graph showing the blood glucose level lowering effect of the IGT ameliorative agent of Example of the invention observed after the first test period in a confirmatory test for blood glucose level lowering effect (oral gavage administration) in which the IGT ameliorative agent was administered at the end of the active phase (dark period).

FIG. 5 is a graph showing the blood glucose level lowering effect of the IGT ameliorative agent of Example of the invention observed after the first test period in a confirmatory test for blood glucose level lowering effect (oral gavage administration) in which the IGT ameliorative agent was administered at the beginning of the active phase (dark period).

FIG. 6 is a graph showing the blood glucose level lowering effect of the IGT ameliorative agent of Example of the invention observed after the latter test period (after the whole testing period) in a confirmatory test for blood glucose level lowering effect (oral gavage administration) in which the IGT ameliorative agent was administered at the end of the active phase (dark period).

FIG. 7 is a graph showing the blood glucose level lowering effect of the IGT ameliorative agent of Example of the invention observed after the latter test period (after the whole testing period) in a confirmatory test for blood glucose level lowering effect (oral gavage administration) in which the IGT ameliorative agent was administered at the beginning of the active phase (dark period).

FIG. 8 is a graph showing the fatty liver ameliorative effect of the IGT ameliorative agent of Example of the invention in a confirmatory test for fatty liver ameliorative effect in which the IGT ameliorative agent was administered at the end of the active phase (dark period).

### DESCRIPTION OF EMBODIMENTS

While the techniques of Patent Literatures 1 to 5 are effective to some extent in reducing an increase in blood glucose level, a more effective agent for the prevention and treatment of IGT has been sought.

The invention relates to an IGT ameliorative agent capable of effectively preventing and treating IGT in diabetic or pre-diabetic humans and animals.

The IGT ameliorative agent of the invention contains as an active ingredient a peak component of partition chromatography of an alcohol extract from gramineous plant seeds. The peak component (the active ingredient of the IGT ameliorative agent of the invention) preferably contains a specific alkylresorcinol mixture containing a plurality of alkylresorcinols represented by general formula (I) shown below. The specific alkylresorcinol mixture is effective in suppressing a blood glucose level increase and ameliorating IGT including diabetes.

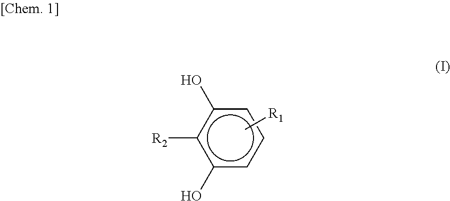

wherein R₁ represents a saturated or unsaturated alkyl group having 15 to 25 carbon atoms; and R₂ represents a hydrogen atom or a methyl group.

Alkylresorcinols are reported to be present in a wide range of plants as resorcinol-lipids that are natural, non-isoterpenoid phenolic, amphiphilic compounds. The plants known as a source of alkylresorcinols include not only gramineous plants (the family Poaceae) include those belonging to the families Anacardiaceae, Ginkgoaceae, Proteaceae, Myrsinaceae, Primulaceae, Myristicaceae, Iridaceae, Araceae, the genus Artemisia of the family Compositae, and Fabaceae. The inventors selected, as a source of an alkylresorcinol, gramineous plants among them because gramineous plants have been intensively studied as an edible active ingredient.

Examples of the gramineous plants for use as a source of alkylresorcinols include, but are not limited to, cereals, such as wheat, durum wheat, rye, triticale, barley, oats, Job's tears (adlay), corn, rice, Japanese millet, foxtail millet, and common millet These plants may be used either individually or in combination of two or more thereof. Preferred of the cereals are those belonging to the genus Triticum, such as wheat and durum wheat, in view of their high activity. Wheat is more preferred. The seeds of the gramineous plant may be of any form. For example, the seeds may be used as such (preferably outer covering such as bran); cut, ground, or powderized; dried; or dried and ground or powderized. Suitable forms of seeds containing outer covering include wheat bran, wheat red dog (the lowest grade of wheat flour), chaff, and rice bran. Seeds with an outer covering are also useful.

The manner for extracting gramineous plant seeds with an alcohol is not particularly limited, including a method in which gramineous plant seeds of various forms described above are immersed, stirred, or refluxed in an alcohol and a supercritical fluid extraction method. In the case of the former method, the extraction temperature is preferably 2° to 100° C.; the extraction time is preferably 30 minutes to 72 hours; and the alcohol is preferably used in an amount of 50 to 2000 parts by mass per 100 parts by mass of the gramineous plant seeds.

Examples of alcohols used for extraction include those that are liquid at room temperature (25° C.), such as lower monohydric alcohols preferably with up to 4 carbon atoms, e.g., methanol, ethanol, n-propanol, isopropyl alcohol, and n-butanol, and polyhydric alcohols, e.g., 1,3-butlene glycol, propylene glycol, and glycerol. Ethanol is preferred in terms of convenience of use and environmental friendliness. Aqueous alcohol containing an aqueous component (e.g., water, pure water, distilled water, tap water, acidic water, alkaline water, or neutral water) may also be used for extraction. The alcohol content of the aqueous alcohol is usually 70 vol % or more, preferably 80 vol % or more, more preferably 90 vol % or more.

The alcohol extract of gramineous plant seeds may be used as an IGT ameliorative agent as it is or concentrated and/or dried or may be purified by known techniques, such as partition chromatography. Any chromatographic technique may be used to carry out partition chromatography to purity the alcohol extract of gramineous plant seeds as long as the active ingredient of the IGT ameliorative agent of the invention (i.e., the specific alkylresorcinol mixture) is collected. Normal phase chromatography using a nonaqueous solvent as a mobile phase is preferred, which is carried out by an appropriately chosen known technique, such as open column chromatography, medium-pressure column chromatography, or high performance liquid chromatography.

Examples of suitable solvents for use as a mobile phase in partition chromatography include alcohols that are liquid at room temperature (25° C.), such as lower monohydric alcohols preferably with up to 4 carbon atoms, e.g., methanol, ethanol, n-propanol, isopropyl alcohol, and n-butanol, and polyhydric alcohols, e.g., 1,3-butlene glycol, propylene glycol, and glycerol; ethers, such as diethyl ether and propyl ether; esters, such as butyl acetate and ethyl acetate; ketones, such as acetone and ethyl methyl ketone; hexane; methylene chloride; acetonitrile; and chloroform. These solvents may be used either individually or in combination of two or more thereof. In the case where two or more solvents are used in combination as a mobile phase in the partition chromatography (purification of the alcohol extract of gramineous plant seeds), the chromatography system may be operated in either an isocratic mode (the mixing ratio of the solvents is constant throughout the chromatographic run) or a gradient mode (the mixing ratio of the solvents is changed during the run).

Any support that is capable of holding and releasing a desired active ingredient may be used in partition chromatography. Commonly used supports include silica gel, polyacrylamide gel, and dextran gel.

The detection wavelength in partition chromatography of the alcohol extract of gramineous plant seeds is 170 to 320 nm, preferably 190 to 280 nm.

Partition chromatography for the purification of the alcohol extract (preferably ethanol extract) of gramineous plant seeds (preferably plants of the genus Triticum) is conveniently carried out by the following methods A or B.

### Method A:

Medium-pressure liquid chromatography using silica gel as a support and a hexane/ethyl acetate mixed solvent as a mobile phase. During the chromatography run, the mobile phase composition is changed in a gradient mode by decreasing the hexane-to-ethyl acetate mixing ratio. The peak component at a detection wavelength of 254 nm is collected.

### Method B:

High-performance liquid chromatography using silica gel as a support and methanol as a mobile phase. The peak component at a detection wavelength of 215 nm is collected.

The content of the active ingredient, i.e., the peak component of the alcohol extract of gramineous plant seeds (specific alkylresorcinol mixture), in the IGT ameliorative agent of the invention is not particularly limited as long as the IGT prophylactic and therapeutic effects are exerted. With a view to ensuring the IGT prophylactic and therapeutic effects, a suitable content of the active ingredient in the IGT ameliorative agent is 50 mass % or more, preferably 70 mass % or more, more preferably 75 mass% or more. The content of the active ingredient (specific alkylresorcinol mixture) may be 100 mass %. That is, the IGT ameliorative agent of the invention may consist solely of the active ingredient (specific alkylresorcinol mixture).

The specific alkylresorcinol mixture, which is the active ingredient of the IGT ameliorative agent of the invention and is preferably contained in the peak component of partition chromatography of the alcohol extract of gramineous plant seeds, will be described in detail. The specific alkylresorcinol mixture contains a plurality of alkylresorcinols represented by general formula (I) shown above.

In formula (I), the C15-C25 saturated alkyl group as represented by R₁ is typically a straight chain saturated alkyl group, such as n-pentadecyl, n-heptadecyl, n-nonadecyl, n-heneicosyl, n-tricosyl, n-pentacosyl, or n-heptacosyl. The saturated alkyl group may also be a branched or a cyclic group. Preferred of these saturated alkyl groups are those having 15 to 23 carbon atoms.

Examples of the C15-C25 unsaturated alkyl group in formula (I) include those corresponding to the above recited C15-C25 saturated alkyl groups. The number and position of the unsaturated bonds in the unsaturated alkyl group are not limited.

R₂ in formula (I) is preferably hydrogen. R₁ is preferably at the para-position with respect to R₂.

Specific examples of the alkylresorcinols that can be present in the specific alkylresorcinol mixture are listed below:
1,3-Dihydroxy-5-n-pentadecylbenzene (C15:0)
1,3-Dihydroxy-5-n-heptadecylbenzene (C17:0)
1,3-Dihydroxy-5-n-nonadecylbenzene (C19:0)
1,3-Dihydroxy-5-n-heneicosylbenzene (C21:0)
1,3-Dihydroxy-5-n-tricosylbenzene (C23:0)
1,3-Dihydroxy-5-n-pentacosylbenzene (C25:0)

The specific alkylresorcinol mixture is preferably exemplified by a mixture containing the six alkylresorcinols described below. The inventors proved these six alkylresorcinols excellent in reducing a blood glucose level increase and effective in prevention and treatment of IGT.
(1) An alkylresorcinol of formula (I) in which R₁ is a C15 saturated or unsaturated alkyl group (hereinafter called AR15)
(2) An alkylresorcinol of formula (I) in which R₁ is a C17 saturated or unsaturated alkyl group (hereinafter called AR17)
(3) An alkylresorcinol of formula (I) in which R₁ is a C19 saturated or unsaturated alkyl group (hereinafter called AR19)
(4) An alkylresorcinol of formula (I) in which R₁ is a C21 saturated or unsaturated alkyl group (hereinafter called AR21)
(5) An alkylresorcinol of formula (I) in which R₁ is a C23 saturated or unsaturated alkyl group (hereinafter called AR23)
(6) An alkylresorcinol of formula (I) in which R₁ is a C25 saturated or unsaturated alkyl group (hereinafter called AR25)

AR15 is preferably an alkylresorcinol of formula (I) in which R₁ is C15 saturated alkyl, and R₂ is hydrogen, i.e., 1,3-dihydroxy-5-n-pentadecylbenzene (C15:0).

AR17 is preferably an alkylresorcinol of formula (I) in which R₁ is C17 saturated alkyl, and R₂ is hydrogen, i.e., 1,3-dihydroxy-5-n-heptadecylbenzene (C17:0).

AR19 is preferably an alkylresorcinol of formula (I) in which R₁ is C19 saturated alkyl, and R₂ is hydrogen, i.e., 1,3-dihydroxy-5-n-nonadecylbenzene (C19:0).

AR21 is preferably an alkylresorcinol of formula (I) in which R₁ is C21 saturated alkyl, and R₂ is hydrogen, i.e., 1,3-dihydroxy-5-n-heneicocylbenzene (C21:0).

AR23 is preferably an alkylresorcinol of formula (I) in which R₁ is C23 saturated alkyl, and R₂ is hydrogen, i.e., 1,3-dihydroxy-5-n-tricocylbenzene (C23:0).

AR25 is preferably an alkylresorcinol of formula (I) in which R₁ is C25 saturated alkyl, and R₂ is hydrogen, i.e., 1,3-dihydroxy-5-n-pentacocylbenzene (C25:0).

The contents of AR15, AR17, AR19, AR21, AR23, and AR25 are preferably in the respective ranges described below with the view of effective prevention and treatment of IGT.

The content of AR15 in the specific alkylresorcinol mixture is preferably 0.1 to 10.0 mass %, more preferably 0.1 to 5.0 mass %, even more preferably 0.5 to 1.5 mass %.

The content of AR17 in the specific alkylresorcinol mixture is preferably 1.0 to 20.0 mass %, more preferably 5.0 to 15.0 mass %, even more preferably 8.0 to 12.0 mass %. The content of AR19 in the specific alkylresorcinol mixture is preferably 25.0 to 40.0 mass %, more preferably 27.5 to 37.5 mass %, even more preferably 30.0 to 35.0 mass %.

The content of AR21 in the specific alkylresorcinol mixture is preferably 40.0 to 55.0 mass %, more preferably 42.5 to 52.5 mass %, even more preferably 45.0 to 50.0 mass %.

The content of AR23 in the specific alkylresorcinol mixture is preferably 1.0 to 15.0 mass %, more preferably 2.5 to 12.5 mass %, even more preferably 5.0 to 10.0 mass %.

The content of AR25 in the specific alkylresorcinol mixture is preferably 0 to 5.0 mass %, more preferably 0 to 2.0 mass %, even more preferably 0 to 1.5 mass %.

The preferred specific alkylresorcinol mixture containing AR15, AR17, AR19,

AR21, AR23, and AR25 may further contain one or more other alkylresorcinols. The other alkylresorcinols include an alkylresorcinol of formula (I) in which R₁ is C27 saturated or unsaturated alkyl (hereinafter called AR27). AR27 is preferably an alkylresorcinol of formula (I) in which R₁ is C27 saturated alkyl, and R₂ is hydrogen, i.e., 1,3-dihydroxy-5-n-heptacosylbenzene (C27:0).

The specific alkylresorcinol mixture may further contain an ingredient other than alkylresorcinols. The content of the other ingredient in the specific alkylresorcinol mixture is preferably not more than 30 mass %.

A preferred example of the specific alkylresorcinol mixture described above is a mixture containing 1.2 mass % 1,3-dihydroxy-5-n-pentadecylbenzene (15:0) as AR15, 10.9 mass % 1,3-dihydroxy-5-n-heptadecylbenzene (C17:0) as AR17, 33.9 mass % 1,3-dihydroxy-5-n-nonadecylbenzene (C19:0) as AR19, 46.4 mass % 1,3-dihydroxy-5-n-heneicosylbenzene (C21:0) as AR21, 7.5 mass % 1,3-dihydroxy-5-n-tricosylbenzene (C23:0) as AR23, and 0.1 mass % 1,3-dihydroxy-5-n-pentacosylbenzene (C25:0) as AR25.

The IGT ameliorative agent of the invention contains a peak component of partition chromatography of the alcohol extract of gramineous plant seeds (the specific alkylresorcinol mixture) and, when needed, various additives, such as carriers and vehicles, and other ingredients that are acceptable for use in pharmaceuticals and health foods such as supplements. The IGT ameliorative agent of the invention can be formulated in a usual manner into preparations of various oral dosage forms, such as tablets, powders, solutions, syrups, granules, and capsules. Examples of the other ingredients that may be incorporated into the IGT ameliorative agent of the invention include ingredients or health foodstuffs having other medicinal effect(s), (e.g., a (raw) ingredient having an inhibitory action on the rise of blood glucose level), various vitamins, crude drugs, and minerals.

The content of the active ingredient (the specific alkylresorcinol mixture) in the IGT ameliorative agent of the invention will be varied appropriately according to the dosage form of the IGT ameliorative agent, the symptoms, age, and the like of a patient or ingester, and so forth. When the IGT ameliorative agent is applied to or ingested by humans, the active ingredient content is preferably adjusted so that a daily dose of the active ingredient may range 0.5 to 5000 mg, preferably 10 to 4500 mg, more preferably 42 to 4200 mg, per adult with a body weight of 60 kg. When the IGT ameliorative agent is applied to or ingested by pet animals, such as dogs and cats, the active ingredient content is preferably adjusted so that a daily dose of the active ingredient may range 0.015 to 150 mg, preferably 0.05 to 150 mg, more preferably 0.13 to 130 mg, per kg of body weight.

The IGT ameliorative agent of the invention may be directly administered to or ingested by humans or animals as a drug or a health food or may be added to or incorporated into food or drink for human and animal consumption (e.g., pet food). Examples of food and drink for human consumption to which the IGT ameliorative agent can be added or incorporated include, but are not limited to, bread, rice, noodles, tablets, confectionery such as candies, soft drinks, juices, and energy drinks. The pet food to which the IGT ameliorative agent can be added or incorporated may be of any type, such as dry, semi-dry, semi-moist, or moist. The manner of adding or incorporating the IGT ameliorative agent into food or drink is not particularly limited. The IGT ameliorative agent can be added directly to a raw material or foodstuff before being processed into food or drink, or added to any intermediate product during processing, or added to a final food or drink product. In the cases where the IGT ameliorative agent of the invention is incorporated into food or drink to be ingested, it may be combined with a foodstuff rich in the specific alkylresorcinol mixture of the invention, for example, wheat bran or whole wheat or rye meal. In such cases, the formulation is adjusted so that the total amount of the specific alkylresorcinol mixture in the finally prepared food or drink may fall within the above described range.

The IGT ameliorative agent or the food or drink of the invention exert excellent ameliorative effect on IGT even in humans and animals who have eaten or been maintained on a high-calorie diet rich in fat and sugar. They may be combined with low-calorie diets or pet foods that are recommended for prevention and treatment of diabetes, in which cases, high ameliorative effect on IGT will be achieved without insulin ingestion or a hypoglycemic agent.

### EXAMPLE

The invention will now be illustrated in greater detail with reference to Example and Tet Examples, but it should be understood that the invention is not limited thereto.

### Example

The specific alkylresorcinol mixture obtained from wheat bran (gramineous plant seeds) by the extraction and purification method described below was used as an IGT ameliorative agent. The specific alkylresorcinol mixture (the IGT ameliorative agent of Examples) as obtained from wheat bran had the following composition:
1,3-Dihydroxy-5-n-pentadecylbenzene (C15:0) 1.2 mass %
1,3-Dihydroxy-5-n-heptadecylbenzene (C17:0) 10.9 mass %
1,3-Dihydroxy-5-n-nonadecylbenzene (C19:0) 33.9 mass %
1,3-Dihydroxy-5-n-heneicosylbenzene (C21:0) 46.4 mass %
1,3-Dihydroxy-5-n-tricosylbenzene (C23:0) 7.5 mass %
1,3-Dihydroxy-5-n-pentacosylbenzene (C25:0) 0.1 mass %

### Extraction and Purification Method:

To wheat bran was added five times the mass of ethanol, and the mixture was stirred at 600 rpm at room temperature for 16 hours to conduct extraction. The mixture was filtered to remove unnecessary matter to give an ethanolic liquid extract. Ethanol was removed by evaporation to give an ethanol extract of wheat bran (gramineous plant seeds). The resulting ethanol extract was purified by medium-pressure chromatography under the conditions below. The peak component appearing during the period of 31 to 36 minutes from the start of elution was collected, and the solvent was removed by evaporation to give the specific alkylresorcinol mixture.

### Conditions of Medium-Pressure Chromatography:

Column: silica gel (Inject Column 3L+Hi-Flash Column 5L; pore size: 60 Å; particle size: 40 μm; available from Yamazen Corp.)
Mobile phase: hexane/ethyl acetate=90/10 (by volume) for 9 minutes; 80/20 for 15 minutes; and 60/40 for 16 minutes
Detection wavelength: 254 nm

The ethanol extract purification in the extraction and purification method described above may be performed by HPLC instead of medium-pressure chromatography. In that case, methanol is added to the ethanol extract to prepare a methanol-added liquid having an ethanol extract concentration of 200 μg/ml. The methanol-added liquid is filtered through a filter with an opening diameter of 0.45 μm. The filtrate is used as a sample for HPLC. Conditions of HPLC are as follows.

### Conditions of HPLC:

Column: silica gel (ODS-80A; particle size: 5 μm; 4.6×250 mm; available from GL Sciences Inc.)
Guard column: ODS-80A (particle size: 5 μm; 4.6×50 mm)
Column temperature: 30° C.
Mobile phase: methanol (100%)
Detection wavelength: 215 nm

### Test Example 1

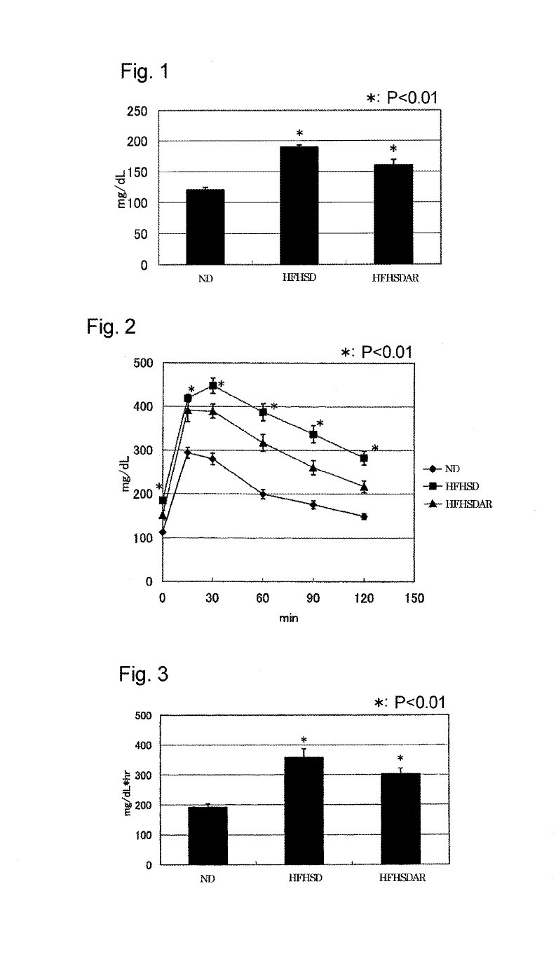

The IGT ameliorative agent of Example was evaluated for the influence on glucose tolerance in mice fed a high-fat high-sucrose diet in accordance with the following test methods. The results obtained are shown in FIGS. 1 to 3, in which ND refers to a group of mice fed a normal diet; HFHSD refers to a group of mice fed a high-fat high-sucrose diet; HFHSDAR refers to a group of mice fed a high-fat high-sucrose diet containing the IGT ameliorative agent (the specific alkylresorcinol mixture) of Example; and the asterisk indicates that a significant difference was observed between ND and HFHSD or HFHSDAR at a significance level of p<0.01.

### Confirmatory Test for Blood Glucose Lowering Effect (Ad Libitum Ingestion)

AIN93M (containing milk casein) and F2HFHSD (both available from Oriental Yeast Co., Ltd.) were provided as a normal diet (ND) and a high-fat high-sucrose diet (HFHSD), respectively. Separately, a high-fat high-sucrose diet having added thereto 0.5 mass % of the IGT ameliorative agent (the specific alkylresorcinol mixture) of Example (HFHSDAR) was prepared. Four-week-old male C57BL/6J Jms Slc mice (from Japan SLC, Inc.) were acclimated to light/dark cycles (12-hour light period and 12-hour dark period; light-on at 0:00, light-off at 12:00) for two weeks. After the acclimation, the mice were divided into three groups: a group fed a normal diet (n=9), a group fed an HFHSD (n=9), and a group fed an HFHSDAR (n=9), and fed ad lib. for 6 weeks under the same light/dark cycles as in the acclimation period. During the ad lib. feeding, a drop of blood was collected from the tail of each mouse occasionally to determine the blood glucose level using a glucometer (Accu-Chek Comfort, from Roche Diagnostics). The average blood glucose level was calculated. The results obtained are shown in FIG. 1.

As is apparent from FIG. 1, the HFHSD-fed group shows a significant increase in fasting blood glucose level as compared with the ND-fed group, clearly proving that intake of an HFHSD can be a cause of hyperglycemia. On the other hand, the group fed the HFHSDAR, which is the HFHSD that can cause hyperglycemia having added thereto a predetermined amount of the IGT ameliorative agent of Example, shows a lower fasting blood glucose level than the HFHSD-fed group. These results lend confirmation to the prophylactic and therapeutic effect of the IGT ameliorative agent of Example on IGT.

### Glucose Tolerance Test

After the confirmatory test for blood glucose lowering effect, the mice were fasted for 4 hours and administered intraperitoneally with a glucose (1 g/kg-b.w.) solution. Blood glucose was monitored at 0 (immediately before glucose administration), 15, 30, 60, 90, and 120 min from the glucose injection in the same manner as in the confirmatory test for blood glucose lowering effect. The AUC_{0-120 min} was calculated according to the method of Wolever, et al. (Wolver T M, Jenkins D J, Jenkins A L, Josse R G, The glycemic index: methodology and clinical implications, Am J Clin Nutr 1991; 54:846-854). The results are shown in FIGS. 2 and 3.

As is apparent from FIGS. 2 and 3, the HFHSD-fed group shows a significant increase in both blood glucose level (FIG. 2) and AUC_{0-120 min} (FIG. 3) as compared with the ND-fed group. It is thus clear that intake of an HFHSD is responsible for hyperglycemia. In contrast, the group fed the HFHSDAR, which is the HFHSD that can cause hyperglycemia having added thereto a predetermined amount of the IGT ameliorative agent of Example, shows a lower blood glucose level and a smaller AUC_{0-120 min} value than the HFHSD-fed group. These results lend confirmation to the prophylactic and therapeutic effect of the IGT ameliorative agent of Example on IGT.

### Test Example 2

The influence of the schedule of administering the IGT ameliorative agent of

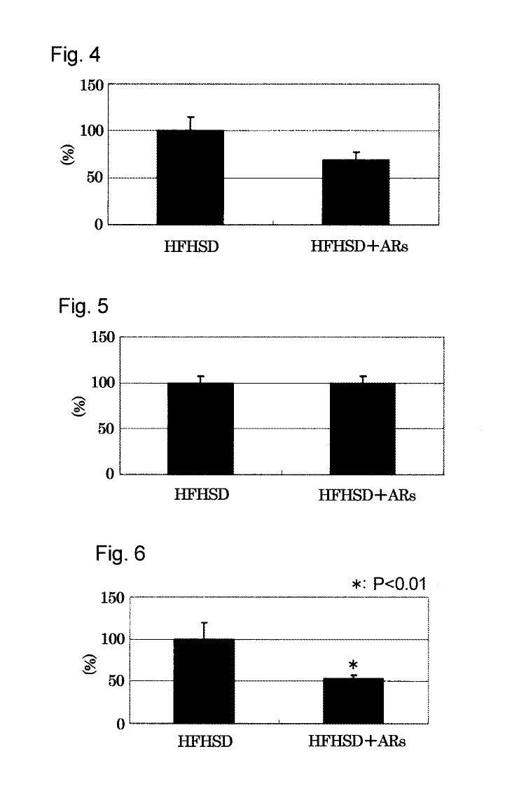

Example on glucose tolerance in mice fed a high-fat high-sucrose diet (HFHSD) was investigated by the tests described below. The results obtained are shown in FIGS. 4 to 8, in which HFHSD refers to a group of mice administered by oral gavage with a blank suspension during the HFHSD feeding period, and HFHSD+ARs refers to a group of mice administered by oral gavage with the IGT ameliorative agent (the specific alkylresorcinol mixture) of Example during the HFHSD feeding period. In FIGS. 6 to 8, the asterisk indicates that a significant difference was observed between HFHSD and HFHSD+ARs at a significance level of p<0.01.

### Confirmatory Test for Blood Glucose Level Lowering Effect (Oral Gavage Administration)

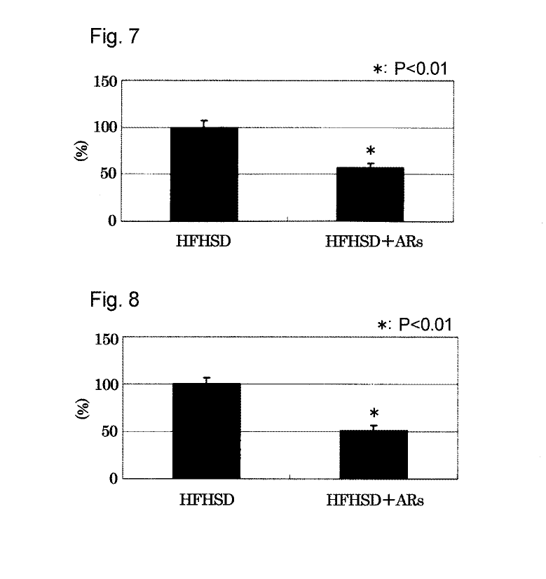

An IGT ameliorative agent suspension was prepared by suspending the IGT ameliorative agent (the specific alkylresorcinol mixture) of Example in ethanol together with olive oil. A high-fat high-sucrose diet (HFHSD) was provided. A blank suspension of olive oil in ethanol was also prepared. Five-week-old male C57BL/6J HamSlc ob/ob mice (from Japan SLC, Inc.) were acclimated to light/dark cycles (12-hour light period and 12-hour dark period; light-on at 0:00, light-off at 12:00) for three weeks. After the acclimation, the mice were divided into four groups: a group administered with the blank suspension at the end of the dark period (ZT=23, 1 hour before light-on), a group administered with the blank suspension at the beginning of the dark period (ZT=13, 1 hour after light-off), a group administered with the IGT ameliorative agent suspension at the end of the dark period (ZT=23), and a group administered with the IGT ameliorative agent suspension at the beginning of the dark period (ZT=13). The test was carried out under the same light/dark cycles as in the acclimation period. The active phase of mice usually corresponds to the dark period. During the testing period, the blank suspension or IGT ameliorative agent suspension was orally given to the mice by gavage. Administration was continued for consecutive four days, interrupted for two days, and again continued for consecutive five days. The dose of the IGT ameliorative agent suspension was 0.025 g of alkylresorcinols per mouse. The mice were fed on the high-fat high-sucrose diet (HFHSD) ad lib. throughout the acclimation period and the testing period. During the ad lib. feeding, the blood glucose level was determined occasionally in the same manner as in the confirmatory test for blood glucose level lowering effect (on mice fed ad libitum), and the average blood glucose level was calculated. The ratio of the average blood glucose level of the mice given the IGT ameliorative agent suspension to that of the mice given the blank suspension, being taken as 100%, is shown in FIGS. 4 through 7. Both FIGS. 4 and 5 represent results obtained after the consecutive four-day administration before the two-day interruption (hereinafter “the first test period”), of which FIG. 4 is the results of administration at the end of the dark period, and FIG. 5 is the results of administration at the beginning of the dark period. Both FIGS. 6 and 7 represent results obtained after the consecutive five-day administration (hereinafter “the latter test period”), i.e., after a total of nine days of administration, of which FIG. 6 is the results of administration at the end of the dark period, and FIG. 7 is the results of administration at the beginning of the dark period.

It is clear from the results of Test Example 1 that intake of FHSD the an HFHSD is responsible for hyperglycemia. It may be said that the mice of Test Example 2 were maintained on a diet leading to hyperglycemia. When such mice were administered by gavage with the IGT ameliorative agent at the end of the active phase (dark period) (ZT=23) in the first test period, they showed a decrease in fasting blood glucose level (see FIG. 4). However, a decrease in fasting blood glucose level was not observed when the IGT ameliorative agent was administered at the beginning of the active phase (dark period) (ZT=13) in the first test period (see FIG. 5). After the entire testing period, the fasting blood glucose level lowered significantly when the IGT ameliorative agent was administered at the beginning of the active phase (ZT=13) (see FIG. 7) as well as when it was administered at the end of the active phase (ZT=23) (see FIG. 6).

It has thus been confirmed that oral gavage administration of the IGT ameliorative agent of Example produces prophylactic and therapeutic effect on IGT. When the administration of the IGT ameliorative agent is done at the beginning of the active phase, outstanding prophylactic and therapeutic effect was not observed in the first test period (see FIG. 5) while the prophylactic and therapeutic effect was obtained in the latter test period (see FIG. 7). When the administration was at the end of the active phase, the prophylactic and therapeutic effect was observed in the first test period (see FIG. 4) and lasted during the latter test period (see FIG. 6). It is seen from these results that oral administration or ingestion of the IGT ameliorative agent of Example is effective in preventing and treating IGT; that the administration or ingestion achieves a desired effect irrespective of the time of administration or ingestion as long as the administration or ingestion lasts for a certain period of time, such as the total testing period including the first and the latter test period; and that higher effect is produced when IGT ameliorative agent of Example is administered or ingested at the end (or in the second half) of the active phase (dark period in the case of mice).

### Confirmatory Test for Ameliorative Effect on Fatty Liver

After the confirmatory test for blood glucose lowering effect (by gavage administration), the mice were dissected, and the amount of triglyceride in the liver was measured. As a result, a decrease in hepatic triglyceride content was slightly observed in the mice administered with the IGT ameliorative agent of Example at the beginning of active phase (dark period) (ZT=13), while a significant decrease in hepatic triglyceride content was observed in the mice administered with the IGT ameliorative agent of Example at the end of the active phase (dark period) (ZT=23) (see FIG. 8). These results reveal that oral administration or ingestion of the IGT ameliorative agent of Example is effective in preventing and treating fatty liver; that the administration or ingestion achieves a desired effect irrespective of the time of administration or ingestion as long as the administration or ingestion lasts for a certain period of time like the total testing period including the first and latter test periods; and that higher effect is produced when IGT ameliorative agent of Example is administered or ingested at the end (or in the second half) of the active phase (dark period in the case of mice).

### Industrial Applicability

The IGT ameliorative agent of the invention effectively prevents or treats diabetes or IGT (pre-diabetes) in humans and animals. Since the active ingredient (alkylresorcinols) of the IGT ameliorative agent of the invention is derived from plants, particularly gramineous plant seeds that have long been eaten, it has high safety and little risk of side effects and yet exhibits excellent ameliorative effect on IGT. Therefore, the IGT ameliorative agent of the invention is extremely beneficial.

1. An impaired glucose tolerance ameliorative agent comprising as an active ingredient a peak component of partition chromatography of an alcohol extract of a gramineous plant seed.

2. The impaired glucose tolerance ameliorative agent according to claim 1, wherein the gramineous plant is a plant belonging to the genus Triticum.

3. The impaired glucose tolerance ameliorative agent according to claim 1, wherein the alcohol is ethanol.

4. The impaired glucose tolerance ameliorative agent according to claim 1, wherein the peak component contains a specific alkylresorcinol mixture comprising a plurality of alkylresorcinols represented by general formula (I): 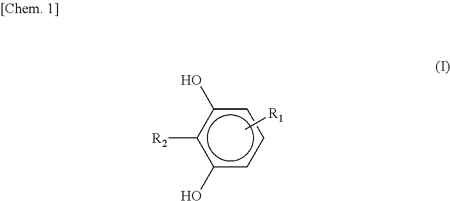
wherein R₁ represents a saturated or unsaturated alkyl group having 15 to 25 carbon atoms; and R₂ represents a hydrogen atom or a methyl group.

5. The impaired glucose tolerance ameliorative agent according to claim 4, wherein the specific alkylresorcinol mixture comprises (1) 0.1 to 10.0 mass % of an alkylresorcinol of formula (I) wherein R₁ is a saturated or unsaturated alkyl group having 15 carbon atoms, (2) 1.0 to 20.0 mass % of an alkylresorcinol of formula (I) wherein R₁ is a saturated or unsaturated alkyl group having 17 carbon atoms, (3) 25.0 to 40.0 mass % of an alkylresorcinol of formula (I) wherein R₁ is a saturated or unsaturated alkyl group having 19 carbon atoms, (4) 40.0 to 55.0 mass % of an alkylresorcinol of formula (I) wherein R₁ is a saturated or unsaturated alkyl group having 21 carbon atoms, (5) 1.0 to 15.0 mass % of an alkylresorcinol of formula (I) wherein R₁ is a saturated or unsaturated alkyl group having 23 carbon atoms, and (6) 0 to 5.0 mass % of an alkylresorcinol of formula (I) wherein R1 is a saturated or unsaturated alkyl group having 25 carbon atoms.

6. The impaired glucose tolerance ameliorative agent according to claim 5, wherein R₁ in formula (I) is at the para-position with respect to R₂.

7. A food or drink for human or animal consumption having incorporated therein the impaired glucose tolerance ameliorative agent according to claim 1.

8. The impaired glucose tolerance ameliorative agent according to claim 2, wherein the alcohol is ethanol.

9. The impaired glucose tolerance ameliorative agent according to claim 2, wherein the peak component contains a specific alkylresorcinol mixture comprising a plurality of alkylresorcinols represented by general formula (I): 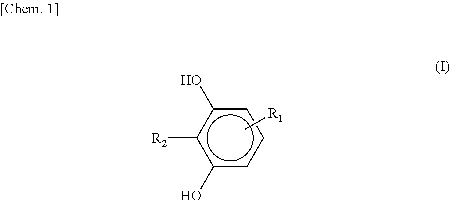
wherein R₁ represents a saturated or unsaturated alkyl group having 15 to 25 carbon atoms; and R₂ represents a hydrogen atom or a methyl group.

10. The impaired glucose tolerance ameliorative agent according to claim 3, wherein the peak component contains a specific alkylresorcinol mixture comprising a plurality of alkylresorcinols represented by general formula (I): 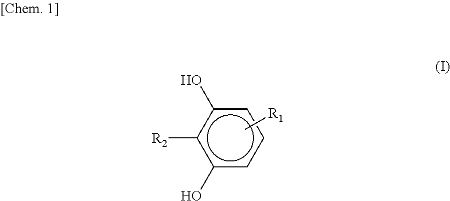
wherein R₁ represents a saturated or unsaturated alkyl group having 15 to 25 carbon atoms; and R₂ represents a hydrogen atom or a methyl group.

11. A food or drink for human or animal consumption having incorporated therein the impaired glucose tolerance ameliorative agent according to claim 2.

12. A food or drink for human or animal consumption having incorporated therein the impaired glucose tolerance ameliorative agent according to claim 3.

13. A food or drink for human or animal consumption having incorporated therein the impaired glucose tolerance ameliorative agent according to claim 4.

14. A food or drink for human or animal consumption having incorporated therein the impaired glucose tolerance ameliorative agent according to claim 5.

15. A food or drink for human or animal consumption having incorporated therein the impaired glucose tolerance ameliorative agent according to claim 6.

16. The impaired glucose tolerance ameliorative agent according to claim 8, wherein the peak component contains a specific alkylresorcinol mixture comprising a plurality of alkylresorcinols represented by general formula (I): 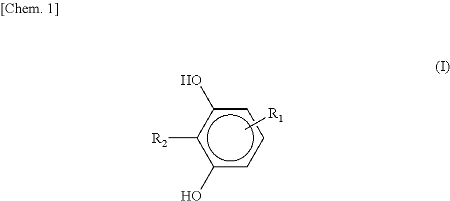
wherein R₁ represents a saturated or unsaturated alkyl group having 15 to 25 carbon atoms; and R₂ represents a hydrogen atom or a methyl group.

17. A food or drink for human or animal consumption having incorporated therein the impaired glucose tolerance ameliorative agent according to claim 8.

18. A food or drink for human or animal consumption having incorporated therein the impaired glucose tolerance ameliorative agent according to claim 9.

19. A food or drink for human or animal consumption having incorporated therein the impaired glucose tolerance ameliorative agent according to claim 10.
